(19) United States
(12) Patent Application Publication
(10) Pub. No.: US 2015/0125899 A1
(43) Pub. Date: May 7, 2015

(54) FLUORESCENCE-ASSISTED COUNTING APPARATUS FOR QUALITATIVE AND/OR QUANTITATIVE MEASUREMENT OF FLUORESCENTLY TAGGED PARTICLES

(71) Applicants: Rebecca Willits, Akron, OH (US); Vivek K. Nagarajan, Akron, OH (US); Amy B. Harkins, St. Louis, MO (US); Shelley D. Minteer, Bountiful, UT (US)

(72) Inventors: Rebecca Willits, Akron, OH (US); Vivek K. Nagarajan, Akron, OH (US); Amy B. Harkins, St. Louis, MO (US); Shelley D. Minteer, Bountiful, UT (US)

(21) Appl. No.: 14/535,716

(22) Filed: Nov. 7, 2014

Related U.S. Application Data
(60) Provisional application No. 61/901,045, filed on Nov. 7, 2013.

(51) Int. Cl. C12Q 1/06; G01N 21/64

(52) U.S. Cl. CPC C12Q 1/06; G01N 21/6486; G01N 2458/00; G01N 2201/062
USPC 435/39; 435/287.9

(57) ABSTRACT

A fluorescence-assisted counting apparatus for quantitative and/or qualitative assessments of a population of fluorescently stained particles in a specimen device retaining fluorescently stained particles, wherein the apparatus includes a detector module and a light source. The detector module includes a lens, a first optical filter and a detector, wherein the detector detects a fluorescence response. The lens includes a field of view taking in at least a portion of the fluorescently stained particles within the specimen device. The light source, further including a second optical filter, shines light in a light path that encompasses at least a portion of the particles.

### CROSS-REFERENCE TO RELATED APPLICATIONS

The present application claims priority to U.S. Provisional Patent Application Ser. No. 61/901,045, filed Nov. 7, 2013, the entirety of which is incorporated herein by reference.

### FIELD OF THE INVENTION

The present invention generally relates to fluorescence-based counting apparatus for quantitative and/or qualitative assessments of the population of fluorescently tagged particles. In some embodiments, the present invention is directed toward a fluorescence-assisted counting apparatus for counting cells or cell organelles within a cell culture specimen device. The fluorescence-assisted counting apparatus includes a specimen device, a detector module, a light source and a shroud. In some embodiments, the light source includes a light source driver.

### BACKGROUND OF THE INVENTION

Cell counting is a measure of the number of cells within a cell culture at a given time. It is an essential parameter when sub-culturing or assessing the effects of experimental treatments on the culture. Cells adhere to the internal side of the membrane, forming a monolayer of cells. Cell counts for monolayers are expressed as cell number per area, typically cm². Cell counting can be broadly classified into automated and manual cell count systems. Of the two, the most commonly used cell counting technique is by means of a manual method.

Automated cell count systems have been implemented using various measurement techniques. Automated cell counters have been based on image analysis, flow (flow cytometers), and electrical impedance (Coulter counters). In recent years, automated cell counting is preferred to manual based cell counting because it offers more reliable results in less time. Also, the use of an automated system significantly reduces user-dependent and concentration-dependent count variance.

Some automated systems such as the NucleoCounter™ (ChemoMetec A/S, Denmark) use Propidium Iodide for labelling the cell to estimate the number of cells based on the detected fluorescence intensity. The use of a fluorescence dye increases the sensitivity and specificity of the system due to the molecular specific binding of the fluorophore. Such systems could be used to study the real-time fluorescence change in the system and relate the measurement to a particular environmental change as the dye characteristics and molecular specificity can also be monitored. In addition, image-based automated cell counters require expensive components such as lenses and cameras and they use sophisticated image processing algorithms to estimate the cell count from the image obtained.

In comparison, a fluorimetric approach, such as the NucleoCounter™, might be cheaper to implement and less bulky than the image-based cell count systems. However, approaches such as the NucleoCounter™ require cell samples provided in a cassette, which is not the same as analyzing an entire cell culture, and cannot be used to culture cells. The cassette consists of flow channels patterned in a way so as to deliver a layer of uniformly distributed cells to the sample chamber.

### SUMMARY OF THE INVENTION

In a first embodiment, the present invention provides a fluorescence-assisted counting apparatus for quantitative and/or qualitative assessments of a population of fluorescently stained particles in a specimen device retaining fluorescently stained particles. The fluorescence-assisted counting apparatus comprises a detector module, including a lens, a first optical filter and a detector. The lens has a field of view which takes in at least a portion of the fluorescently stained particles in the specimen device and the detector detects a fluorescence response. A light source shines light, in a light path that encompasses at least a portion of the fluorescently stained particles, to excite the fluorescently stained particles to produce a fluorescence response and the light source includes a second optical filter.

In a second embodiment, the present invention provides a fluorescence-assisted cell counting apparatus as in the first embodiment, wherein the specimen device includes a cell growth membrane with fluorescently stained particles adhered thereto and wherein the fluorescently stained particles are cells or cell organelles.

In a third embodiment, the present invention provides a fluorescence-assisted cell counting apparatus as in the first or second embodiments, wherein the cell growth membrane includes at least one gas-permeable membrane.

In a fourth embodiment, the present invention provides a fluorescence-assisted cell counting apparatus as in any of the first through third embodiments, wherein the specimen device includes one or more sidewalls and an opposed wall. The opposed wall provides an enclosed environment for the cells or cell organelles.

In a fifth embodiment, the present invention provides a fluorescence-assisted cell counting apparatus as in any of the first through fourth embodiments, wherein the opposed wall is a cell growth membrane.

In a sixth embodiment, the present invention provides a fluorescence-assisted cell counting apparatus as in any of the first through fifth embodiments, wherein the specimen device includes one or more ports to provide access to the interior of the specimen device.

In a seventh embodiment, the present invention provides a fluorescence-assisted cell counting apparatus as in any of the first through sixth embodiments, wherein the specimen device includes a cell growth membrane comprised of two gas-permeable membranes.

In an eighth embodiment, the present invention provides a fluorescence-assisted cell counting apparatus as in any of the first through seventh embodiments, wherein the two gas-permeable membranes comprise a cell culture treated with polystyrene membrane.

In a ninth embodiment, the present invention provides a fluorescence-assisted cell counting apparatus as in any of the first through eighth embodiments, wherein the two gas-permeable membranes are parallel to one another.

In a tenth embodiment, the present invention provides a fluorescence-assisted cell counting apparatus as in any of the first through ninth embodiments, wherein the gas-permeable membranes are at least 75 μm thick and at least 2 mm apart.

In an eleventh embodiment, the present invention provides a fluorescence-assisted cell counting apparatus as in any of the first through tenth embodiments, wherein the two parallel gas-permeable membranes provide a growth area of at least 50 cm².

In a twelfth embodiment, the present invention provides a fluorescence-assisted cell counting apparatus as in any of the first through eleventh embodiments, wherein the detector module is placed at a first working distance, L.

In a thirteenth embodiment, the present invention provides a fluorescence-assisted cell counting apparatus as in any of the first through twelfth embodiments, wherein the detector is selected from the group consisting of UV-VIS, biolumen or fiber optic cable.

In a fourteenth embodiment, the present invention provides a fluorescence-assisted cell counting apparatus as in any of the first through thirteenth embodiments, wherein the detector is a silicon photodiode.

In a fifteenth embodiment, the present invention provides a fluorescence-assisted cell counting apparatus as in any of the first through fourteenth embodiments, wherein the lens is a spherical lens.

In a sixteenth embodiment, the present invention provides a fluorescence-assisted cell counting apparatus as in any of the first through fifteenth embodiments, wherein the first optical filter filters out the excitation spectra within the detector module.

In a seventeenth embodiment, the present invention provides a fluorescence-assisted cell counting apparatus as in any of the first through sixteenth embodiments, wherein the light source is placed at a second working distance, L2.

In an eighteenth embodiment, the present invention provides a fluorescence-assisted cell counting apparatus as in any of the first through seventeenth embodiments, wherein the light source produces a light path, P, illuminating the cell growth membrane.

In a nineteenth embodiment, the present invention provides a fluorescence-assisted cell counting apparatus as in any of the first through eighteenth embodiments, wherein the light source is an LED or plurality of LEDs.

In a twentieth embodiment, the present invention provides a fluorescence-assisted cell counting apparatus as in any of the first through nineteenth embodiments, wherein the light source is selected from a light emitting diode (LED) or a laser diode with diffuser.

In a twenty-first embodiment, the present invention provides a fluorescence-assisted cell counting apparatus as in any of the first through twentieth embodiments, wherein the light source includes a light source driver to maintain a stable current to the light source.

In a twenty-second embodiment, the present invention provides a fluorescence-assisted cell counting apparatus as in any of the first through twenty-first embodiments, wherein the light source driver is selected from an LED driver or laser diode driver.

In a twenty-third embodiment, the present invention provides a method for counting cells in a fluorescence-assisted counting apparatus for quantitative and/or qualitative assessments of a population of fluorescently stained particles in a specimen device retaining fluorescently stained particles, wherein the method comprises the steps of: staining the particles with a fluorescence dye to provide stained particles, exposing the stained particles to light suitable to cause a fluorescence response in the specimen device, measuring the intensity of the fluorescence response and correlating the intensity of the fluorescence response to a concentration of particles.

In a twenty-fourth embodiment, the method for counting cells in a fluorescence-assisted counting apparatus provided for in any of the first through twenty-third embodiments, wherein the particles are cells or cell organelles.

### BRIEF DESCRIPTION OF THE DRAWINGS

FIG. 1 is front elevational view is an embodiment of the fluorescence-assisted counting apparatus in accordance with the present invention;

FIG. 2 is front elevational view is a further embodiment of the fluorescence-assisted counting apparatus in accordance with the present invention;

FIG. 3 is a front elevation view of the detector module of the fluorescence-assisted counting apparatus in accordance with the present invention;

FIG. 4 is a plan view depicting the outer limits of the field of view V tangential to the sidewalls of the cell growth membrane in accordance with the present invention;

FIG. 5 is a plan view depicting the field of view V tangential to the outer corners of the cell growth membrane footprint and wherein the field of view V completely engulfs the cell growth membrane in accordance with the present invention.

### DETAILED DESCRIPTION OF ILLUSTRATIVE EMBODIMENTS

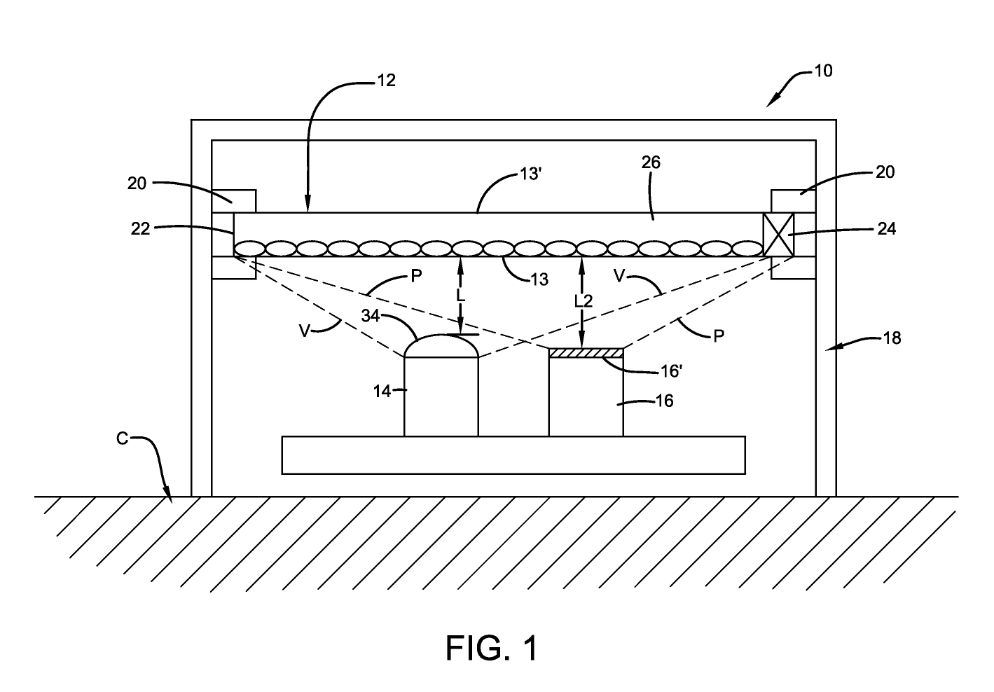

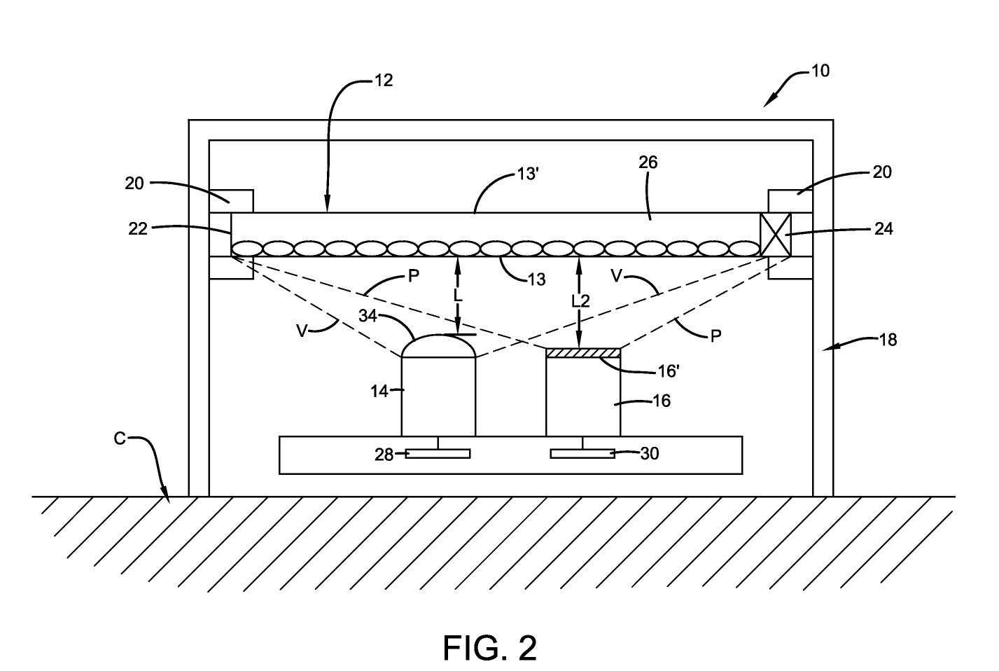

With reference to FIGS. 1-2, the present invention provides a fluorescence-assisted counting apparatus 10 for quantitative and/or qualitative assessments of the population of fluorescently tagged particles within a specimen device 12. The fluorescence-assisted counting apparatus 10 comprises a detector module 14 and a light source 16. In some embodiments, the light source 16 provides a stable and consistent light intensity. In some embodiments, the detector module 14 is connected to a microprocessor 28. In some embodiments, the fluorescence-assisted counting apparatus 10 and specimen device 12 are enclosed in a shroud 18 to omit ambient light during use.

The specimen device 12 includes any particle that can be stained with a fluorescence dye to provide a fluorescence response when subjected to a excitation wavelength. In some embodiments, the particles may include cells, cell organelles, microbeads and quantum dots stained to provide a fluorescence response.

In the disclosure that follows, the focus is on the assessment of cells within a cell culture specimen device serving as the specimen device 12, but it will be appreciated that the same apparatus and concepts can be employed to assess any fluorescently tagged particle.

Thus, in some embodiments, the apparatus 10 is employed to count cell or cell organelles within a cell culture specimen device 12. The cell culture specimen device 12 includes at least one cell growth membrane 13 to which cells adhere. The cells (or cell organelles thereof) in the cell culture specimen device 12 are stained with a fluorescent dye and are exposed to light from the light source 16 providing an excitation wavelength causing a fluorescence response by the cells. This fluorescence response is detected by the detector module 14 in order to count cells (in a quantifying operation) or record either cell growth or cell death (in a qualifying operation).

In some embodiments, the fluorescence-assisted counting apparatus 10 qualifies cell growth or cell death. Cell growth is defined for present purposes as growth of the number of live cells in a cell culture specimen device 12, i.e., relating to the population of the cells, not their size. In such embodiments, the device can be used to count fluorescently labeled microparticles/beads or any fluorescently labeled cellular component.

Similarly, cell death is defined as a decrease in the number of live cells within a cell culture specimen device 12. The counting apparatus 10 can be used to simply observe a change in the number of live cells, either an increase, indicating cell growth, or a decrease, indicating cell death. In such situation, cells are not actually being “counted” per se, but a change in fluorescence is being observed by the apparatus thus relating to either cell growth (evidenced by an increase of fluorescence) or cell death (evidenced by a decrease of fluorescence). When live cells undergo death, the fluorescence disappears and the fluorescence intensity resulting from the exposure to the light source 16 decreases. Thus, for observing cell death, the cells can be dyed and the fluorescence reading taken over time to observe that, over time, the fluorescence intensity decreases and thus cells are dying. For observing cell growth, it will be necessary to expose the cells again to the dye before taking a fluorescence reading so that newly produced cells can uptake the dye and their fluorescence response can contribute to the fluorescence intensity.

In some embodiments, the fluorescence-assisted counting apparatus 10 quantifies the concentration of cells in an unknown cell culture (or “test sample”) that is placed within the cell culture specimen device 12. In such embodiments, a standard curve is first created for a given type of cell using a given type of fluorescent dye. Known concentrations of cells within a cell culture specimen device 12 (the concentration identified through other known methods distinct from the present invention) are stained with a fluorescent dye, and the cell culture specimen device 12 is positioned relative to the apparatus 10 to be exposed to light from the light source 16. The detector module 14 of the fluorescence-assisted counting apparatus 10 measures the fluorescence intensity of these different concentrations of cells in cell culture specimen devices 12, and a graph is plotted showing fluorescence intensity as a function of cell concentration. A best-fit line is determined. After creation of this fluorescence intensity versus cell concentration curve, the cell concentration of a test sample (of the same type of cells) in a cell culture specimen device 12 can be determined by staining the cells therein with the same fluorescent dye, placing the cell culture specimen device 12 in counting apparatus 10, exposing the cells of the cell culture membrane 13 to light from the light source 16, and obtaining a fluorescence intensity from the detector module 14. The fluorescence-assisted counting apparatus 10 will measure the fluorescence intensity of the test sample, which can then be compared with the standard curve to determine the cell concentration.

Referring to FIGS. 1-2, in some embodiments, the fluorescence-assisted counting apparatus 10 is enclosed in a shroud 18 to omit ambient light during counting. The emitted light from the light source 16 must be completely filtered from ambient light in order to accurately and consistently measure a fluorescent intensity. In such embodiments, the shroud 18 must completely encompass the fluorescence-assisted counting apparatus 10. In some embodiments, the shroud 18 provides support to the fluorescence-assisted counting apparatus 10. In such embodiments, the shroud 18 is placed on a countertop, C, or any other surface of the like. In some embodiments, the shroud 18 includes receipts 20 for the securing of the cell culture specimen device 12 therein, opposite the detector module 14 and light source 16. The shroud 18 fits over the detector module 14 and the light source 16 and is sized so as to hold the cell culture specimen device 12 at a working distance from the light source 16 and detector module 14, so that at least 25π% of the entire surface area of the membrane to which the cells adhere is illuminated by the light from the light source 16 and is within the field of view of the detector module 14. In other embodiments, the entire surface area of the membrane to which the cells adhere is lit by the light from the light source 16 and is within the field of view of the detector module 14.

In other embodiments, the fluorescence-assisted counting apparatus 10 may be enclosed in a dark room or other receptacle to completely omit ambient light during counting. In such embodiments, the cell culture specimen device 12 will be placed on top of or under the detector module 14 and light source 16 in order to measure a fluorescent intensity.

The fluorescence-assisted counting apparatus 10 offers several advantages for monitoring and screening pathogens or toxic substances over other conventional counting devices. Some advantages include simulating the physiological in vivo response, which provides a means to study the effect of specific cellular functions due to test analytes and lowers the cost per test, as compared to the time and money required to raise and maintain an animal. Further, cells suspended within the cell culture membrane 12 or adhered to the cell growth membrane 13 are completely protected against contamination and physical stresses, by providing membranes that are impermeable to fungi and bacteria, while providing sufficient gas exchange.

In its broadest form, the specimen device 12 is a suitable support or container for a particle that can be stained with a fluorescence dye to provide a fluorescence response when subjected to a excitation wavelength. In some embodiments, the specimen device 12 presents the particles in a single layer so that the fluorescence response of each particle is not hidden behind another.

In some embodiments as already focused upon above, the specimen device is a cell culture specimen device 12. A cell culture specimen device 12 includes at least one cell growth membrane 13 to which cells adhere, comprised of any material that is transparent to light, particularly the light of the light source 16, such that the fluorescence response of the cells can be observed and/or recorded by the detector module 14. The cells in the cell culture specimen device 12 adhere to a surface (or face) of the cell growth membrane 13. In such embodiments, the cell culture will form a monolayer of cells.

The cell culture specimen device 12 comprising the cell growth membrane 13 allows for cell growth in conditions closer to an in vivo environment. Some advantages include portability, robustness, and a stable internal sterile environment, which enables the use and transport of cell cultures for monitoring or analysis without compromising the sterility of the internal environment. The cell culture specimen device 12 containing a cell culture or biological sample is able to be sealed and shipped without destroying the integrity of the sample adhered to the cell growth membrane 13. Further, the cell culture specimen device 12 comprising the cell growth membrane 13 can be used for either fluorescence or confocal microscopy to monitor expression of reporter genes in transfected cells, death by apoptosis or necrosis, and cell activation.

In some embodiments, when the cell culture specimen device 12 is rectangular, the cell growth membrane 13 is rectangular in shape. In other embodiments, when the cell culture specimen device 12 is circular, the cell growth membrane 13 is circular in shape.

In some embodiments, the cell growth membrane 13 is gas-permeable to maintain the aerobic metabolism of the cells by permitting optimal oxygen and carbon dioxide exchange with the atmosphere. In some embodiments, one or more sidewalls 22 and an opposed wall join the cell growth membrane 13 to provide an enclosed environment for the cells. In some embodiments, the opposed wall is a cell growth membrane 13′.

In some embodiments, in order to permit optical oxygen and carbon dioxide exchange, the gas-permeable membrane has a thickness from 75 μm or more to 3000 μm or less.

Referring to FIG. 1, in some embodiments, the cell growth membrane 13 includes two opposed gas-permeable membranes, cell growth membrane 13 and cell growth membrane 13′. In such embodiments, the two gas-permeable membranes are placed at a distance from 2000 μm or more to 3000 μm or less apart from one another.

In some embodiments one or more sidewalls 22 join the opposed cell growth membranes 13, 13′ to provide and enclosed environment for the cells. In some embodiments, the cell culture specimen device 12 includes one or more ports, as at 24, to provide access to the interior 26 of the cell culture specimen device 12 for the introduction of cells, fluorescent beads, dyes or solutions that are used for a cell culture.

The cell growth membrane 13 can be selected from the group consisting of culture treated polystyrene membranes, any optical transparent membrane on which cells can be cultured, or the like.

In preferred embodiments, the cell culture specimen device 12 includes two parallel gas-permeable membranes made up of culture treated polystyrene membranes, (e.g. OptiCell™ Nunc membranes, OPNM). In such embodiments, the cell culture specimen device 12 includes two resealing access ports 24 to provide access to a sterile fluid path and a closed cell growth environment. The two parallel gas-permeable membranes are about 75 μm in thickness and are about 2 mm apart, thereby providing a growth area of about 50 cm².

In some embodiments, a cell growth membrane 13 selected will have the same size and format as an OPNM. In other embodiments, the thickness of a cell growth membrane from 75 μm or more to 3000 μm or less.

The cell cultures within the cell culture specimen device 12 of the fluorescence-assisted counting apparatus 10 are stained with a fluorescent dye to detect the fluorescence response. The fluorescent dye is selected based on the cell (or particle in broader terms) being stained—notably, the dye must be suitable for achieving the staining of the cell (or cell subpart in cases where a cell subpart is of interest). Similarly, the light source 16 used must be suitable for providing the excitation wavelength that produces the fluorescence response in the dye employed. The use of a fluorescent dye increases the sensitivity and specificity of the system due to the molecular specific binding of the fluorophore. Several different fluorescence dyes are commercially available. In some embodiments, the fluorescent dye is selected from any fluorescent dye used for labelling the cell nucleus.

The selected fluorophore will label the cell nuclei of the cell culture sample and therefore, the number of cells can be estimated based on the detected fluorescence response. In some embodiments, a fluorophore may be selected from group of any type of fluorophore in the visible spectra.

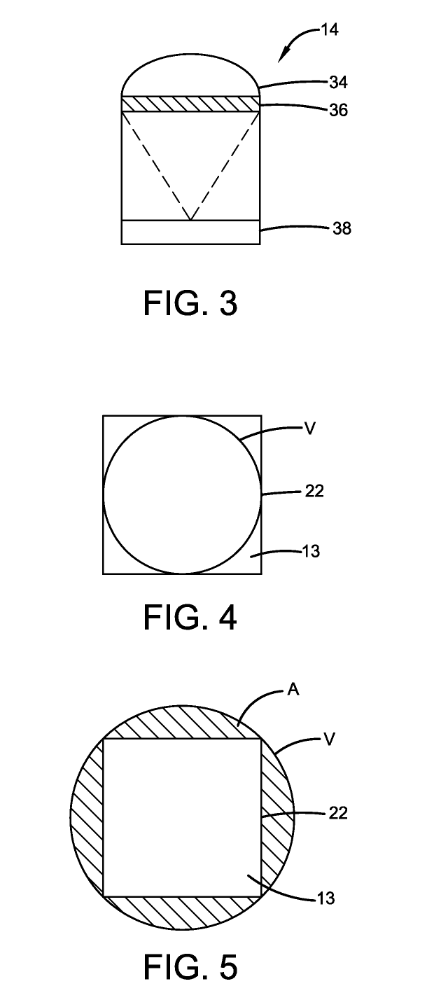

The fluorescence-assisted counting apparatus 10 includes a detector module 14. With reference to FIG. 3, the detector module 14 includes a lens 34, an optical filter 36 and a detector 38. In some embodiments, the detector module 14 is further connected to a microprocessor 28, as shown in FIG. 2. The choice and design of the detector module 14 may be dependent upon the total amount of optical power incident on the detector 38.

The fluorescence response emitted from cells in the cell growth membrane 13 is captured by the lens 34, which aids in focusing the emitted signal onto the detector 38 within the detector module 14. In order to detect the fluorescence response of the cell culture, the detector module 14 is placed at a working distance, L, from the cell culture specimen device 12, as seen in FIGS. 1-2. The working distance L induces a field of view, V, for the detector module 14. In order to measure the fluorescence response, the detector module 14 should be placed at a working distance L that is appropriate to produce an adequate field of view V to take in the cells on the cell growth membrane.

Referring to FIG. 1, in some embodiments, the lens 34 is a spherical lens. In some embodiments, a spherical lens 34 is used with a rectangular cell culture specimen device 12. In such embodiments, the field of view V will completely engulf the cell growth membrane 13 footprint and the field of view V will be cone-shaped. In some embodiments, the field of view V may not completely engulf the cell growth membrane 13 footprint, however, the cell culture specimen device 12 is useful nonetheless to observe either cell growth or cell death, though perhaps quantifications would suffer.

As shown in FIG. 4, in some embodiments, the detector module 14 is placed at a working distance L wherein the outer limits of the field of view V is tangential to the sidewalls 22 defining the cell growth membrane 13. The field of view V will not recognize negligible cell culture concentrations found within the outer portions of the cell growth membrane 13. In such embodiments, the field of view V will illuminate approximately 75% of the cell growth membrane 13 surface area in order to measure cell culture concentrations.

As shown in FIG. 5, in other embodiments, the field of view V is tangential to the outer corners of the cell growth membrane 13 footprint when the cell culture specimen device 12 is rectangular. In such embodiments, the field of view V completely engulfs the cell growth membrane 13 for illumination. In such embodiments, the detector module 14 will measure cell concentrations illuminated on the entire surface area of the cell growth membrane 13, as well as absent area A.

In other embodiments, the cell culture specimen device 12 may be circular. In such embodiments, the field of view V will completely illuminate to preferably match the footprint (surface area) of the cell growth membrane 13 footprint when a spherical lens is used. In other embodiments, the field of view V may be pyramidal depending upon the type of lens used. In such embodiments, the field of view V will completely illuminate the cell growth membrane 13 without measuring negative area.

In some embodiments, the lens 34 is a PCX, a camera, or lens system of the like.

With reference to FIG. 3, an optical filter 36 is placed between the lens 34 and the detector 38, wherein the field of view is projected by the lens 34 onto the detector 38 through the optical filter 36. The optical filter 36 is provided to filter out the excitation spectra from the emission spectra expected from the fluorescence dye employed. Thus, the emission spectrum, as perceived by the detector 38, is not negatively impacted by the excitation spectra. The filtering reduces the amount of fluorescence signal onto the detector 38. The use of a long-pass filter on the detector 38 end will increase the fluorescence signal, enabling better detection of lower concentrations.

In some embodiments, the optical filter 36 is an emission filter. Emission filters allows for the transmission of the emitted fluorescence from the cell growth membrane 13 into the detector module 14, while rejecting the excitation spectra from the light source 16. The selection of the excitation filter is such that the filter allows for the transmission of the excitation spectra and rejects wavelengths in the emission spectra.

In some embodiments, the optical filter 36 is an optical bandpass filter. In other embodiments, the optical filter 36 may be selected from the group consisting of any filter which allows for the transmission of excitation spectra and rejects wavelengths of the emission spectra.

In some embodiments, the optical filter 36 is a filter wheel placed between the lens 34 and the detector 38. A filter wheel allows for easy removal and installation of multiple filters within the detector module to obtain the appropriate filter for filtering out the excitation spectra from the emission spectra expected from the fluorescence dye employed. In some embodiments, a user can switch between filters on the filter wheel manually, or in other embodiments, operation of the filter wheel is automated.

The detector 38 detects the fluorescence response emitted from the cells within the cell growth membrane 13. After passing through an optical filter 36, the fluorescent response is projected onto the detector 38. The total optical power incident onto the detector 38 depends on the total fluorescence signal emitted from the samples within on the cell growth membrane 13. The projection of the lens 34 onto the detector 38 is then used to obtain the total fluorescence signal incident onto the detector 38. The detector 38 produces a signal to be compared to the standard curve to compare for quantification, or can observe a change in signal over time to evidence growth/death. The fluorescence signal will increase with cell growth and will decrease upon cell death.

In some embodiments, the detector 38 is a silicon photodiode. In other embodiments, the detector may be selected from the group consisting of UV-VIS, biolumen, fiber optic cable, APD (avalanche photodiode), PMT (photomultiplier tube), cell-phone camera, tablet PC, or the like.

As shown in FIG. 2, in some embodiments, the detector module 14 is connected to a microprocessor 28. The standard curve to determine cell concentration is programmed within the microprocessor 28, wherein the fluorescence response is used to determine the concentration of the cell culture directly from the microprocessor 28 data. In short, a human need not compare fluorescence intensity to a standard curve, but rather the data relevant to the standard curve is loaded into the microprocessor 28, which can simply provide a resultant cell count by calculating the cell count based on the standard curve data.

The fluorescence-assisted counting apparatus 10 includes a light source 16. In some embodiments, the light source 16 includes an optical filter 16′, as shown in FIGS. 1-2. The optical filter 16′ is placed in front of the light source 16 to limit the spectra of the illumination to the excitation spectra of the fluorescent dye, such that only wavelengths within the excitation spectra are transmitted.

In some embodiments, the light source 16 includes a light source driver 30, as shown in FIG. 2. The light source 16 induces the stained cell culture adhered to the cell growth membrane 13 to fluoresce in order to facilitate the fluorescence-based counting. The power spectrum of the light source 16 determines the shape and intensity of the fluorescence emission signal.

The fluorescence response of the cell culture depends upon the wavelength and optical power of the incident light emitted from the light source 16. Broadband sources such as, halogen lamps, xenon arc lamps and mercury arc lamps can be used to obtain spectra of the fluorescence emission signals. For fluorescence-based cell count, the intensity of the fluorescence response at wavelength of maximum emission is used for cell count estimation.

The light source 16 is placed at the working distance, L₂, from the cell culture specimen device 12, as seen in FIGS. 1-2. The light source 16 is placed at working distance L₂ to produce a light path, P, onto the cell growth membrane 13. In some embodiments, the light source 16 and the detector module 14 are equidistant to the cell culture specimen device 12.

In some embodiments, the light source 16 is a monochromatic light source. The fluorescence response from the cell growth membrane 13 depends upon the wavelength and optical power of the incident light from the light source 16. In some embodiments, the light source 16 includes an adjustable power source to induce fluorescence. In some embodiments, the light source 16 is a light emitting diode (LED), in other embodiments, a laser, a fluorescent lamp or broadband source of the like.

In preferred embodiments, the light source 16 is an LED. Two main parameters, brightness and wavelength of maximum emission, determine the choice of LED to be used in the fluorescence-assisted counting apparatus 10. LEDs provide high output power at the wavelength of maximum emission. In some embodiments, the LED can be selected from a super bright yellow LED or any LED selected based on the excitation spectra of the dye.

In some embodiments, the light source 16 includes at least one LED, in other embodiments, two or more LEDs, and still in other embodiments, multiple LEDs may be chosen to provide uniform illumination. By increasing the number of LEDs present in the light source 16, the current supply (or the inflow of electrons) increases, thereby increasing the number of photons emitted from the cell growth membrane 13, which will produce a stronger fluorescence response.

Referring to FIG. 2, in some embodiments, the light source 16 includes a light source driver 30 to maintain a stable current. A stable current supply is required for providing power to the light source 16, as the performance of light source 16 depends upon the stability of the current supplied to it. The light source driver 30 supplies a linear current to the light source 16 providing temperature stability, i.e. the current remains stable over a temperature range thereby maintaining the brightness of the light source 16 over that temperature range. The light source driver 30 further maintains a stable current supplied to the light source 16, thereby producing a constant output.

In some embodiments, the light source driver 30 is adjustable. By increasing the current supply (or the inflow of electrons) into the light source 16, the number of photons emitted from the cell growth membrane 13 will increase, thereby increasing the strength of the fluorescence response.

In some embodiments, the light source 16 is an LED light source and includes a light source driver to maintain a stable current. A stable current supply is required for providing power to the LED light source, as the performance of the LED light source depends upon the stability of the current supplied to it. The light source driver supplies a linear current to the LED light source providing temperature stability, i.e. the current remains stable over a temperature range thereby maintaining the brightness of the LED light source over that temperature range. The light source driver further maintains a stable current supplied to the LED light source, thereby producing a constant output.

The fluorescence-assisted counting apparatus 10 includes an optical filter 16′ placed over the light source 16, as seen in FIGS. 1-2. The optical filter 16′ provides for the transmission of excitation spectra and rejects wavelengths of the emission spectra. In some embodiments, the optical filter 16′ is an optical bandpass filter. In other embodiments, the optical filter 16′ may be selected from the group consisting of a short-pass filter for excitation and a long-pass filter for emission spectra.

In some embodiments, the apparatus 10 includes a plurality of (two or more) detectors, each having an optical filter suitable for filtering for a specific emission spectra. This allows for the use of two dyes in one specimen device 12, and thus allows for the study of multiple types of particles, cells, cell organelles, microbeads, quantum dots, etc.

In some embodiments, the light source 16 and the one or more detector modules 14 are provided in a shroud 18 and form a complete unit that can selectively receive specimen devices 12. The specimen device 12 would be inserted into an opening provided for feeding the specimen device 12 from the exterior to the interior of the shroud 18, and the specimen device so loaded would be positioned appropriately for excitation and observing of a fluorescence response as described herein. With portable cell culture supports becoming more popular, this is a particularly beneficial concept, as it allows these portable cell culture supports (as specimen devices) to be selectively loaded into the counting apparatus. The apparatus itself would also be beneficially portable in other embodiments.

The method of using the fluorescence-assisted cell culture apparatus 10 for counting cells first requires creating a standard curve. Known concentrations of cells are stained with a fluorescent dye, placed within the cell culture specimen device 12 and exposed to light from the light source 16. The fluorescence-assisted counting apparatus 10 will measure the fluorescence intensity of the cell culture. The fluorescence intensity of the known cell concentrations is then plotted on a graph and a best-fit line is determined. After creation of this fluorescence intensity versus cell concentration curve, the cell concentration of a test sample in a cell culture specimen device 12 can be determined by staining the cells therein with the same fluorescent dye, placing the cell culture specimen device 12 in counting apparatus 10, exposing the cells of the cell culture specimen device 12 to light from the light source 16, and obtaining a fluorescence intensity from the detector module 14. The fluorescence-assisted counting apparatus 10 will measure the fluorescence intensity of the test sample, which can then be compared with the standard curve to determine the cell concentration.

Next, using standard live cell staining procedures, the cell culture is stained with a fluorescent dye. The selected dye is dependent upon the light source 16 chosen. The fluorescent dye is cell specific, and therefore the type of dye chosen will be selected based on the type of test sample, as well as the type of light source 16 used. The stained cell cultures are then loaded onto the cell growth membrane 13. In such embodiments, the labeled cell solution is injected into the cell culture specimen device 12 via the one or more ports 24.

After the cell culture has been dyed and loaded onto the cell growth membrane 13, the cell growth membrane 13 is exposed to light from the light source 16 to induce the cell culture to fluoresce. The fluorescence-assisted counting apparatus 10 will then measure the fluorescence intensity of the cell culture. Once the fluorescence intensity has been measured, it is recorded.

Once the fluorescent intensity of the unknown cell culture sample is finally recorded, is then compared with the standard curve previously produced to obtain the cell culture count.

In light of the foregoing, it should be appreciated that the present invention significantly advances the art by providing a portable fluorescence-assisted counting apparatus for counting cells that is structurally and functionally improved in a number of ways. While particular embodiments of the invention have been disclosed in detail herein, it should be appreciated that the invention is not limited thereto or thereby inasmuch as variations on the invention herein will be readily appreciated by those of ordinary skill in the art. The scope of the invention shall be appreciated from the claims that follow.

What is claimed is:

1. A fluorescence-assisted counting apparatus for quantitative and/or qualitative assessments of a population of fluorescently stained particles in a specimen device retaining fluorescently stained particles, the fluorescence-assisted counting apparatus comprising:
a detector module, including a lens, a first optical filter and a detector, said lens having a field of view taking in at least a portion of said fluorescently stained particles in said specimen device, said detector detecting a fluorescence response; and
a light source shining light in a light path that encompasses at least a portion of the fluorescently stained particles to excite said fluorescently stained particles to produce a fluorescence response, said light source including a second optical filter.

2. The fluorescence-assisted cell counting apparatus of claim 1, wherein said specimen device includes a cell growth membrane with fluorescently stained particles adhered thereto, wherein said fluorescently stained particles are cells or cell organelles.

3. The fluorescence-assisted cell counting apparatus of claim 2, wherein said cell growth membrane includes at least one gas-permeable membrane.

4. The fluorescence-assisted cell counting apparatus of claim 3, wherein said specimen device includes one or more sidewalls and an opposed wall to provide an enclosed environment for the cells or cell organelles.

5. The fluorescence-assisted cell counting apparatus of claim 4, wherein said opposed wall is a cell growth membrane.

6. The fluorescence-assisted cell counting apparatus of claim 5, wherein said specimen device includes one or more ports to provide access to the interior of said specimen device.

7. The fluorescence-assisted cell counting apparatus of claim 2, wherein said specimen device includes a cell growth membrane comprised of two gas-permeable membranes.

8. The fluorescence-assisted cell counting apparatus of claim 7, wherein said two gas-permeable membranes comprise a cell culture treated with polystyrene membrane.

9. The fluorescence-assisted cell counting apparatus of claim 8, wherein said two gas-permeable membranes are parallel to one another.

10. The fluorescence-assisted cell counting apparatus of claim 1, where said detector is selected from the group consisting of UV-VIS, biolumen or fiber optic cable.

11. The fluorescence-assisted cell counting apparatus of claim 1, wherein said detector is a silicon photodiode.

12. The fluorescence-assisted cell counting apparatus of claim 1, wherein said first optical filter filters out the excitation spectra within said detector module.

13. The fluorescence-assisted cell counting apparatus of claim 1, wherein said light source produces a light path, P, illuminating said cell growth membrane.

14. The fluorescence-assisted cell counting apparatus of claim 1, wherein said light source is an LED or plurality of LEDs.

15. The fluorescence-assisted cell counting apparatus of claim 1, wherein said light source is selected from a light emitting diode (LED) or a laser diode with diffuser.

16. The fluorescence-assisted cell counting apparatus of claim 15, wherein said light source includes a light source driver to maintain a stable current to said light source.

17. The fluorescence-assisted cell counting apparatus of claim 16, wherein said light source driver is selected from an LED driver or laser diode driver.

18. A method for counting cells in a fluorescence-assisted counting apparatus for quantitative and/or qualitative assessments of a population of fluorescently stained particles in a specimen device retaining fluorescently stained particles, the method comprising the steps of:
staining said particles with a fluorescence dye to provide stained particles,
exposing said stained particles to light suitable to cause a fluorescence response in said specimen device,
measuring the intensity of the fluorescence response; and
correlating the intensity of the fluorescence response to a concentration of particles.

19. The method for counting cells in a fluorescence-assisted counting apparatus of claim 18, wherein said particles are cells or cell organelles.
